(19) United States
(12) Patent Application Publication
(10) Pub. No.: US 2023/0321455 A1
(43) Pub. Date: Oct. 12, 2023

(54) SYSTEMS AND METHODS FOR PROVIDING ALPHA BURST BRAIN STIMULATION

(71) Applicant: Wave Neuroscience, Inc., Newport Beach, CA (US)

(72) Inventor: James William Phillips, Fountain Valley, CA (US)

(21) Appl. No.: 18/298,306

(22) Filed: Apr. 10, 2023

Related U.S. Application Data
(60) Provisional application No. 63/362,796, filed on Apr. 11, 2022.

(51) Int. Cl. A61N 2/00; A61B 5/369

(52) U.S. Cl. CPC A61N 2/006; A61B 5/369

(57) ABSTRACT

A method of treating a subject that includes measuring at least one alpha burst associated with the subject's brain, determining at least one intrinsic frequency of the subject's brain based on the at least one measured alpha burst, selecting a stimulation frequency based on the at least one intrinsic frequency of the subject's brain, and providing a stimulation treatment at the stimulation frequency close to a head of the subject to influence at least one alpha burst parameter of the subject's brain.

### CROSS-REFERENCE TO RELATED APPLICATIONS

This application claims priority to and the benefit of U.S. Provisional Patent Application No. 63/362,796, titled “SYSTEMS AND METHODS FOR PROVIDING ALPHA BURST BRAIN STIMULATION” and filed on Apr. 11, 2022, the entire contents of which is hereby incorporated by reference herein.

### TECHNICAL FIELD

This specification relates to brain stimulation and, in particular, to providing alpha burst brain stimulation to a human subject.

### BACKGROUND

The electrical activity of a person's brain can be viewed through measurement of the person's electroencephalogram (EEG). The EEG measures voltage fluctuations resulting from current flow within neurons. Alpha waves are neural oscillations that generally occur at one or more intrinsic frequencies between 8-13 Hz. Alpha waves indicate idleness, or a lack of concerted activity in the brain. However, alpha waves are often predominant in individuals who have excellent concentration abilities, are calm, focused, and relaxed. As such, alpha activity is believed to play a central role in information processing and control.

### SUMMARY

At least one aspect of the present disclosure is directed to a method of treating a subject. The method includes measuring at least one alpha burst associated with the subject's brain, determining at least one intrinsic frequency of the subject's brain based on the at least one measured alpha burst, selecting a stimulation frequency based on the at least one intrinsic frequency of the subject's brain, and providing a stimulation treatment at the stimulation frequency close to a head of the subject to influence at least one alpha burst parameter of the subject's brain.

In some embodiments, applying the stimulation treatment close to the head of the subject to influence the at least one alpha burst parameter includes optimizing at least one of an alpha frequency variability (AFV) and an alpha prevalence (AP) of the subject. In some embodiments, optimizing the AFV of the subject includes minimizing the AFV of the subject. In some embodiments, optimizing the AP of the subject includes maximizing the AP of the subject. In some embodiments, measuring the at least one alpha burst associated with the subject's brain includes collecting an electroencephalogram (EEG) of the subject. In some embodiments, selecting the stimulation frequency includes calculating an average frequency of a plurality of intrinsic frequencies. In some embodiments, the average frequency is a weighted average corresponding to the duration of each alpha burst associated with the plurality of intrinsic frequencies.

In some embodiments, the method includes determining a value of the at least one alpha burst parameter based on the at least one measured alpha burst, comparing the value of the at least one alpha burst parameter to an expected value for the at least one alpha burst parameter, selecting, based on a result of the comparison, a tuning frequency, and providing a stimulation treatment at the tuning frequency close to a head of the subject to influence at least one alpha burst parameter of the subject's brain. In some embodiments, the method includes measuring at least one second alpha burst associated with the subject's brain, determining at least one second intrinsic frequency of the subject's brain based on the at least one second alpha burst, selecting a second stimulation frequency based on the at least one second intrinsic frequency of the subject's brain, providing a second stimulation treatment at the second stimulation frequency close to a head of the subject to influence at least one alpha burst parameter of the subject's brain.

Another aspect of the present disclosure is directed to a system for providing treatment to a subject. The system includes at least one stimulation source, at least one memory storing computer-executable instructions, and at least one processor for executing the instructions stored on the memory. Execution of the instructions programs the at least one processor to perform operations that include measuring at least one alpha burst associated with the subject's brain, determining at least one intrinsic frequency of the subject's brain based on the at least one measured alpha burst, selecting a stimulation frequency based on the at least one intrinsic frequency of the subject's brain, and providing, via the at least one stimulation source, a stimulation treatment at the stimulation frequency close to a head of the subject to influence at least one alpha burst parameter of the subject's brain.

In some embodiments, applying the stimulation treatment close to the head of the subject to influence the at least one alpha burst parameter includes optimizing at least one of an alpha frequency variability (AFV) and an alpha prevalence (AP) of the subject. In some embodiments, optimizing the AFV of the subject includes minimizing the AFV of the subject. In some embodiments, optimizing the AP of the subject includes maximizing the AP of the subject. In some embodiments, measuring the at least one alpha burst associated with the subject's brain includes collecting an electroencephalogram (EEG) of the subject. In some embodiments, selecting the stimulation frequency includes calculating an average frequency of a plurality of intrinsic frequencies. In some embodiments, the average frequency is a weighted average corresponding to the duration of each alpha burst associated with the plurality of intrinsic frequencies.

In some embodiments, execution of the instructions further programs the at least one processor to perform operations that include determining a value of the at least one alpha burst parameter based on the at least one measured alpha burst, comparing the value of the at least one alpha burst parameter to an expected value for the at least one alpha burst parameter, selecting, based on a result of the comparison, a tuning frequency, and providing, via the at least one stimulation source, a stimulation treatment at the tuning frequency close to a head of the subject to influence at least one alpha burst parameter of the subject's brain. In some embodiments, execution of the instructions further programs the at least one processor to perform operations that include measuring at least one second alpha burst associated with the subject's brain, determining at least one second intrinsic frequency of the subject's brain based on the at least one second alpha burst, selecting a second stimulation frequency based on the at least one second intrinsic frequency of the subject's brain, and providing, via the at least one stimulation source, a second stimulation treatment at the second stimulation frequency close to a head of the subject to influence at least one alpha burst parameter of the subject's brain.

Another aspect of the present disclosure is directed to a device for use in treating a subject. The device includes a means for applying a stimulation treatment to a head of the subject. The means for applying the stimulation treatment comprises a first processor that controls the application of the stimulation treatment. The first processor or a second processor moves at least one of an alpha frequency variability (AFV) of the subject's brain below a pre-defined AFV threshold using the magnetic field and an alpha prevalence (AP) of the subject's brain above a pre-defined AP threshold using the magnetic field.

In some embodiments, the pre-defined AFV and AP thresholds are derived from AFV and AP values corresponding to a plurality of healthy individuals.

Another aspect of the present disclosure is directed to a method of treating a subject. The method includes measuring at least one alpha burst associated with the subject's brain, determining at least one intrinsic frequency of the subject's brain based on the at least one measured alpha burst, selecting a stimulation frequency based on the at least one intrinsic frequency of the subject's brain, generating, via at least one magnetic source, a magnetic field having the stimulation frequency, and applying the magnetic field close to a head of the subject to influence at least one alpha burst parameter of the subject's brain.

In some embodiments, applying the magnetic field close to the head of the subject to influence the at least one alpha burst parameter includes optimizing at least one of an alpha frequency variability (AFV) and an alpha prevalence (AP) of the subject. In some embodiments, optimizing the AFV of the subject includes minimizing the AFV of the subject. In some embodiments, optimizing the AP of the subject includes maximizing the AP of the subject. In some embodiments, measuring the at least one alpha burst associated with the subject's brain includes collecting an electroencephalogram (EEG) of the subject. In some embodiments, selecting the stimulation frequency includes calculating an average frequency of a plurality of intrinsic frequencies. In some embodiments, the average frequency is a weighted average corresponding to the duration of each alpha burst associated with the plurality of intrinsic frequencies.

In some embodiments, the method includes determining a value of the at least one alpha burst parameter based on the at least one measured alpha burst, comparing the value of the at least one alpha burst parameter to an expected value for the at least one alpha burst parameter, selecting, based on a result of the comparison, a tuning frequency, generating, via the at least one magnetic source, a magnetic field having the tuning frequency, and applying the magnetic field close to the head of the subject to influence the at least one alpha burst parameter of the subject's brain. In some embodiments, the method includes measuring at least one second alpha burst associated with the subject's brain, determining at least one second intrinsic frequency of the subject's brain based on the at least one second alpha burst, selecting a second stimulation frequency based on the at least one second intrinsic frequency of the subject's brain, generating, via the at least one magnetic source, a second magnetic field having the second stimulation frequency, and applying the second magnetic field close to the head of the subject to influence the at least one alpha burst parameter of the subject's brain.

Another aspect of the present disclosure is directed to a system for providing treatment to a subject. The system includes at least one magnetic source, at least one memory storing computer-executable instructions, and at least one processor for executing the instructions stored on the memory. Execution of the instructions programs the at least one processor to perform operations that include measuring at least one alpha burst associated with the subject's brain, determining at least one intrinsic frequency of the subject's brain based on the at least one measured alpha burst, selecting a stimulation frequency based on the at least one intrinsic frequency of the subject's brain, generating, via at least one magnetic source, a magnetic field having the stimulation frequency, and applying the magnetic field close to a head of the subject to influence at least one alpha burst parameter of the subject's brain.

In some embodiments, applying the magnetic field close to the head of the subject to influence the at least one alpha burst parameter includes optimizing at least one of an alpha frequency variability (AFV) and an alpha prevalence (AP) of the subject. In some embodiments, optimizing the AFV of the subject includes minimizing the AFV of the subject. In some embodiments, optimizing the AP of the subject includes maximizing the AP of the subject. In some embodiments, measuring the at least one alpha burst associated with the subject's brain includes collecting an electroencephalogram (EEG) of the subject. In some embodiments, selecting the stimulation frequency includes calculating an average frequency of a plurality of intrinsic frequencies. In some embodiments, the average frequency is a weighted average corresponding to the duration of each alpha burst associated with the plurality of intrinsic frequencies.

In some embodiments, execution of the instructions further programs the at least one processor to perform operations that include determining a value of the at least one alpha burst parameter based on the at least one measured alpha burst, comparing the value of the at least one alpha burst parameter to an expected value for the at least one alpha burst parameter, selecting, based on a result of the comparison, a tuning frequency, generating, via the at least one magnetic source, a magnetic field having the tuning frequency, and applying the magnetic field close to the head of the subject to influence the at least one alpha burst parameter of the subject's brain. In some embodiments, execution of the instructions further programs the at least one processor to perform operations that include measuring at least one second alpha burst associated with the subject's brain, determining at least one second intrinsic frequency of the subject's brain based on the at least one second alpha burst, selecting a second stimulation frequency based on the at least one second intrinsic frequency of the subject's brain, generating, via the at least one magnetic source, a second magnetic field having the second stimulation frequency, and applying the second magnetic field close to the head of the subject to influence the at least one alpha burst parameter of the subject's brain.

Another aspect of the present disclosure is directed to a device for use in treating a subject. The device includes a means for applying a magnetic field to a head of the subject. The means for applying the magnetic field comprises a first processor that controls the application of the magnetic field. The first processor or a second processor moves at least one of an alpha frequency variability (AFV) of the subject's brain below a pre-defined AFV threshold using the magnetic field and an alpha prevalence (AP) of the subject's brain above a pre-defined AP threshold using the magnetic field.

In some embodiments, the pre-defined AFV and AP thresholds are derived from AFV and AP values corresponding to a plurality of healthy individuals.

### BRIEF DESCRIPTION OF THE DRAWINGS

FIG. 1 illustrates a block diagram of a system for determining and optimizing the alpha burst parameters of a human subject in accordance with aspects described herein;

FIG. 2 illustrates a plot of example data collected by measurement devices in accordance with aspects described herein;

FIG. 3 illustrates a flow diagram of a method for determining the alpha burst parameters of a human subject in accordance with aspects described herein;

FIG. 4 illustrates an example alpha parameter report in accordance with aspects described herein;

FIG. 5 illustrates an example alpha parameter report in accordance with aspects described herein; and

FIG. 6 illustrates an example computing device.

### DETAILED DESCRIPTION

As discussed above, the electrical activity of a person's brain can be viewed through measurement of the person's EEG. The EEG measures voltage fluctuations resulting from current flow within neurons. Alpha waves are neural oscillations that generally occur at one or more intrinsic frequencies between 8-13 Hz. These alpha waves are most evident on an EEG recording when a person is at rest, awake, with eyes closed. Alpha waves indicate idleness, or a lack of concerted activity in the brain. However, alpha waves are often predominant in individuals who have excellent concentration abilities, are calm, focused, and relaxed. As such, alpha activity is believed to play a central role in information processing and control.

In some embodiments, Intrinsic Alpha Frequency (IAF) represents the dominant brainwave frequency in the alpha EEG band (e.g., 8-13 Hz). IAF varies between individuals, with each person evincing a different dominant alpha frequency. The dominant alpha frequency tends to vary over the long term, with IAF dropping approximately ¼ Hz per decade after age 50. In some embodiments, a low alpha frequency is associated with cognitive problems and mental disorders. For example, dementia and Alzheimer's disease patients tend to have an alpha rhythm well below 8.0 Hz. In some embodiments, cognitive difficulty occurs when a person's IAF drops below 8.0 Hz.

In some embodiments, IAF tends to vary for a person over the short term. This intra-subject Alpha Frequency Variability (AFV) reflects different alpha networks kicking in dependent on task demands. In some embodiments, AFV reflects fluctuations in moment-to-moment performance, and changes based on the person's mental state. AFV is one reason for the large bandwidth seen in spectral analysis of EEG waveforms as a whole. However, IAF is often clearer and shows less variability when the EEG is analyzed in small temporal increments. Alpha activity tends to be represented in bursts, with short duration alpha bursts or spindles evident. For example, in some embodiments alpha bursts last from approximately 0.5 sec to approximately 2.0 sec in duration or longer. Some individuals spend the majority of their waking hours in an alpha burst state, while others may have no discernable alpha burst(s) at all.

In some embodiments, the intrinsic frequency of other EEG bands (e.g., outside 8-13 Hz) exhibit variability between bursts. For example, in some embodiments when a person transitions to certain stages of sleep, sleep spindles are seen on an EEG recording having an intrinsic frequency between 11-16 Hz. In some embodiments, these bursts or spindles are classified as beta bursts. In some embodiments, other intrinsic frequencies of other EEG bands exhibit similar burst-like behavior.

In some embodiments, the IAF recorded during individual alpha bursts varies from burst to burst. For example, a slight variability (e.g., up to 0.2 Hz) is common in human subjects. However, in some embodiments a significant variability (e.g., above 0.25 Hz) is associated with poor cognitive processing or a potential mental disorder. Therefore, the optimal brain state, or optimal brain health, corresponds to minimal variability in IAF from one alpha burst to another.

In some embodiments, the prevalence of alpha activity is indicative of optimal (or preferred) mental health. For example, in some embodiments a person who is calm, relaxed, and/or able to focus on single tasks will have alpha bursts that are longer and have higher amplitude than someone who is less able to focus and/or who may suffer from stress or anxiety. In addition to AFV, the duration and time interval between alpha bursts is an indicator of a person's brain state (or brain health). In some embodiments, a brain's mental state and health is improved when a greater percentage of time is spent in an alpha burst (e.g., relative to time spent outside an alpha burst). This percentage is referred to as the Alpha Prevalence (AP) herein.

Alternating or pulsed brain stimulation, in which the stimulation pulse frequency is equal to the intrinsic frequency of an EEG band (e.g., the alpha band) has been shown to have an effect on symptoms of major depression and other disorders. However, due to the brain's natural variability in alpha frequency, it is difficult to determine and/or accurately provide stimulation at the exact intrinsic frequency of human subjects. For example, in some embodiments the value of the intrinsic frequency is different depending on the time, duration, and alpha burst activity of the recording.

Accordingly, systems and methods for determining and optimizing alpha burst parameters are provided herein. In at least one embodiment, the alpha burst parameters include the AFV and AP of a person's brain. In some embodiments, the alpha burst parameters are optimized using targeted, personalized, brain stimulation. The AFV and AP are used as part of a report (or analysis) to estimate a person's mental state or capacity. For example, in some embodiments a low AFV and/or a high AP provides an indication of an improved ability to concentrate on activities, whereas a high AFV and/or a low AP provides an indication of difficulty focusing and concentrating.

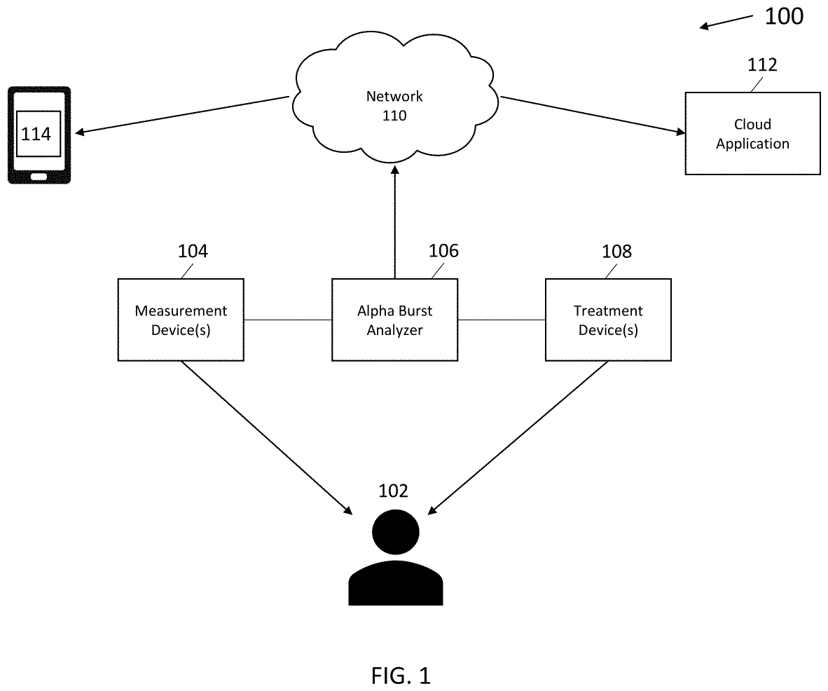

FIG. 1 is a functional block diagram of a system 100 for determining and optimizing the alpha burst parameters of a human subject 102. In some embodiments, the system 100 includes at least one measurement device 104, an alpha burst analyzer 106, and at least one treatment device 108.

The measurement device 104 is configured to measure (or collect) data corresponding to the electrical activity the subject's brain. For example, in some embodiments the measurement device 104 is configured to collect an EEG waveform. In some embodiments, the measurement device 104 is capable of measuring or collecting other types of data including at least one of an electrocardiogram (ECG) recording, an MM image, a Brain Score, an EKG recording, a SPECT scan, a PET scan, x-ray, CT scan, Ultrasound, mammogram, Fluoroscopy, arthrogram, myelogram, DEXA bone density scan, body temperature, respiratory rate, heart rate, blood pressure, blood oxygen saturation, Complete Blood Count, basic metabolic panel, comprehensive metabolic panel, lipid panel, liver panel, thyroid stimulating hormone, hemoglobin A1C Prothrombin time, blood enzyme tests, blood clotting test, urinalysis, cultures, applanation tonometry, corneal topography, Fluorescein angiogram, slit-lamp exam image, retinal tomography, visual acuity testing, visual field test results, mental health assessment, behavioral health assessment, psychiatric assessment, athletic performance measurement, academic performance measurement, intelligence test result, self-assessment, demographics, and personality profile.

In some embodiments, the alpha burst analyzer 106 receives the data collected by the measurement device 104. The alpha burst analyzer 106 is configured to process the collected data to identify one or more alpha bursts. In some embodiments, the alpha burst analyzer 106 is configured to analyze the identified alpha bursts to determine the subject's alpha burst parameters (e.g., AFV and AP).

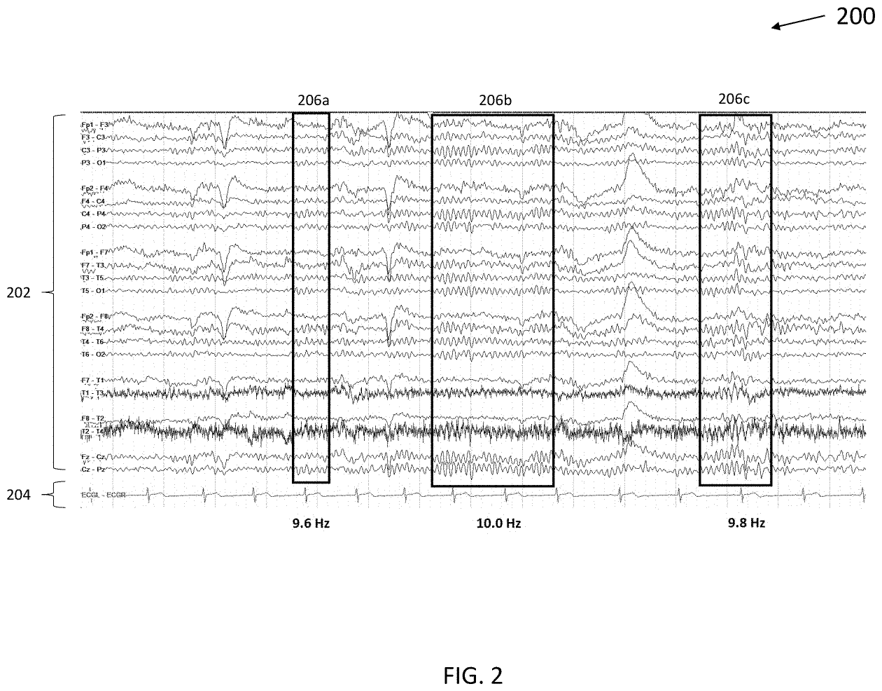

FIG. 2 is a plot 200 of example data collected by the measurement device 104 and provided to the alpha burst analyzer 106. In one embodiment, the data includes an EEG waveform 202 and an ECG waveform 204. In the illustrated example, the EEG waveform 202 includes 20 channels and the ECG waveform 204 includes 1 channel. The boxed-in areas show alpha bursts, where alpha EEG activity is most evident throughout the brain. The first alpha burst 206a is 0.65 sec in duration with an IAF of 9.6 Hz. The second alpha burst 206b is 2.35 sec in duration with an IAF of 10.0 Hz. The third alpha burst 206c is 1.38 sec in duration with an IAF of 9.8 Hz. In one example, the recording of the EEG waveform 202 is 15.0 sec in duration, and the total time spent in alpha burst(s) was 4.7 sec, or approximately 4.7/15.0=31%. Therefore, the AP is 31%.

In some embodiments, the alpha burst analyzer 106 corresponds to an application or applications configured to run on one or more of a smart phone, a tablet computer, a smart watch, a laptop computer, a desktop computer, or other similar devices. In some embodiments, the alpha burst analyzer 106 is configured to communicate with the measurement device 104 via a wired or wireless connection (e.g., Bluetooth, WiFi, USB, etc.). In some embodiments, the alpha burst analyzer 106 is configured to communicate with a cloud application 112 and a user application 114. The alpha burst analyzer 106 communicates with the applications 112, 114 via a network 110, such as the Internet, for example.

In some embodiments, the alpha burst analyzer 106 forwards the results of the alpha burst analysis and/or the original data (e.g., collected by the measurement device 104) to the cloud application 112 and the user application 114. In some embodiments, the alpha burst analyzer 106 forwards the results of the alpha burst analysis and/or the original data to the cloud application 112 and the cloud application 112 forwards the data (or a portion of the data) to the user application 114. The results of the alpha burst analysis are provided to the subject 102, the subject's physician, or other medical professionals via the applications 112, 114. In some embodiments, the cloud application 112 stores the results in one or more databases (e.g., cloud-based databases). In some embodiments, the cloud application 112 is configured to perform further processing or analysis on the alpha burst data or forward the data to another application for further processing/analysis. In some embodiments, the alpha burst analyzer 106 is included in the cloud application 112 or the user application 114.

The treatment device 108 is configured to provide treatment (e.g., brain stimulation) to the subject's brain based on the alpha burst parameters of the user 102. In some embodiments, the treatment device 108 is configured to provide magnetic brain stimulation to optimize at least one alpha burst parameter, as described in greater detail below. In some embodiments, the treatment corresponds to a treatment plan (or treatment settings) provided by the alpha burst analyzer 106, the cloud application 112, or the user application 114. In some embodiments, the treatment device 108 includes at least one magnetic source (e.g., electromagnet or permanent magnets) configured to provide at least one magnetic field having a stimulation (or therapeutic) frequency. In some embodiments, the treatment device 108 includes at least one rotating magnet. In some embodiments, the treatment device 108 corresponds to one or more devices, mechanisms, and techniques described in U.S. Pat. No. 9,308,387, titled “Systems and Methods for Neuro-EEG Synchronization Therapy” and granted on Apr. 13, 2016, which is hereby incorporated by reference herein in its entirety.

In some embodiments, the treatment device 108 includes at least one stimulation source (e.g., magnetic source, electric source, etc.). The at least one stimulation source provides, at least in part, a stimulation treatment at one or more stimulation frequencies. In some embodiments, the treatment device 108 provides brain stimulation using transcranial electrical stimulation. In some embodiments, the treatment device 108 provides brain stimulation using focused ultrasound. In some embodiments, the treatment device 108 provides brain stimulation using functional Near Infrared Spectroscopy (fNIRS). In some embodiments, the treatment device 108 provides brain stimulation using sensory stimulation. For example, in some embodiments the treatment device 108 provides sensory stimulation including flashing light, sound, video, or touch.

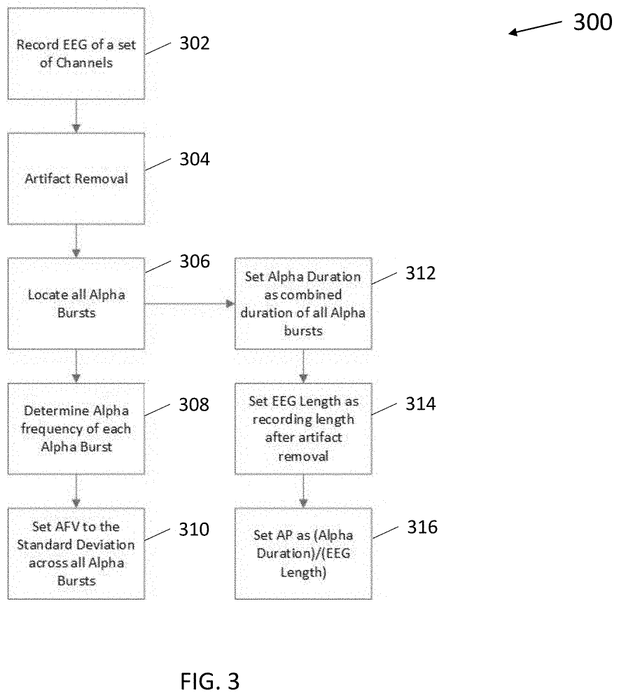

FIG. 3 is a flow diagram of a method 300 for determining the alpha burst parameters of a human subject (e.g., subject 102). In some embodiments, the method 300 is configured to be carried out by the measurement device(s) 104 and the alpha burst analyzer 106 of FIG. 1.

At step 302, an EEG recording is collected via the measurement device 104. In some embodiments, the measurement device 104 corresponds to an EEG cap with at least one electrode (e.g., scalp electrodes). In some embodiments, the measurement device 104 corresponds to a dry-lead EEG headset system, an implantable EEG system, or an EEG system that uses percutaneous leads. The EEG recording represents the subject's brain activity across at least one EEG channel (e.g., 1, 10, 20, 30, etc.), and the recording extends for a period of time. In some embodiments, the EEG recording corresponds to a set (or subset) of EEG channels. For example, in some embodiments the set comprises EEG channels pertaining to electrodes from one or more areas of the scalp that are of interest, EEG channels where the signal exceeds a pre-specified signal to noise ratio, or EEG channels with pre-specified spectral characteristics, such as theta power being below a predetermined threshold. In some embodiments, the set comprises a single EEG channel. In some embodiments, the EEG recording is collected while the subject's eyes are closed, because alpha activity is most prevalent when the eyes are closed and the subject is relaxed. Once collected, the EEG recording is provided to the alpha burst analyzer 106.

At step 304, the alpha burst analyzer 106 is configured to remove artifacts capable of corrupting the EEG recording. For example, in some embodiments artifacts include eye blinks, eyelid flutter, facial contractions such as frowning, general movement such as in adjusting in a chair, heartbeat artifacts, and other movements that are capable of corrupting the EEG recording. In some embodiments, artifact removal methods include frequency-based methods, like linear filtering, correlation methods, in which wavelets are correlated with the signal to look for specific patterns that indicate artifacts, or non-linear filtering. In certain embodiments, artifact removal is optional.

At step 306, the alpha burst analyzer 106 is configured to locate alpha bursts in the EEG recording. In some embodiments the alpha burst analyzer 106 labels each identified alpha burst. At step 308, the alpha burst analyzer 106 is configured to determine the alpha frequencies of each alpha burst in the EEG recording. Several approaches for locating alpha bursts and determine the corresponding alpha frequencies are provided herein.

In some embodiments, a first approach includes the use of a moving filter to find the energy at the IAF for each channel's EEG recording at each time point. The energy at the IAF for all channels in the set is averaged together to find the overall energy. The alpha bursts all have a dominant frequency in the alpha range. In some embodiments, the overall energy is compared to a threshold to determine whether or not a time point is part of an alpha burst.

In some embodiments, a second approach includes dividing each channel's EEG recording into segments of a prespecified duration. A Fourier Transform is computed for each segment. The frequency where the maximum energy of the signal occurs is determined to be the IAF for each channel's segment. In some embodiments, a segment is determined to be part of an alpha burst when a prespecified number of channels in the recording (e.g., at least 50%) have a maximum energy that exceeds a predetermined threshold. In some embodiments, each alpha burst includes one segment or multiple consecutive segments. The overall burst energy is the average maximum energy across all channels at the respective IAF.

In some embodiments, a third approach includes moving a sliding window across each EEG channel and computing a Fourier Transform for each window. The frequency where the channel's maximum energy of the signal occurs is considered the alpha frequency for that window. The overall energy value corresponds to the average of the maximum energy across all channels. In some embodiments, an alpha burst is considered as the section of the EEG recording where the overall energy value exceeds a pre-determined threshold.

In some embodiments, a fourth approach includes a wavelet that is used for correlation with each channel's EEG signal. For example, in some embodiments a Morlet wavelet with a specified fundamental frequency in the alpha EEG band is correlated with each channel's signal to generate a waveform that is highest in amplitude during sections where the dominant EEG frequency matches the Morlet wavelet frequency. In some embodiments, a single Morlet wavelet is used, with a frequency equal to the average alpha frequency of the channel's EEG recording. In some embodiments, multiple Morlet wavelets are tested, each with a different frequency in the alpha EEG band. The overall Morlet correlation is determined as the average correlation across all channels in the set. In some embodiments, a burst is defined as the section of the EEG recording where the overall Morlet correlation exceeds a pre-determined threshold. The alpha frequency of the burst is the wavelet frequency corresponding to the maximum energy, averaged across all channels.

At step 310, the alpha burst analyzer 106 is configured to determine the AFV of the set of EEG channels. In some embodiments, the AFV is determined (or calculated) using the alpha frequency estimates of each burst (e.g., from step 308). For example, in some embodiments one or more alpha bursts are determined for the EEG recording, with each alpha burst having an IAF. In some embodiments, the AFV is calculated on a per channel basis. The channel AFV is determined as the standard deviation of the IAF of every burst in the channel's EEG recording. In some embodiments, the channel AFV is calculated as the weighted standard deviation of all bursts in the channel's EEG recording, where the weight of each burst IAF is proportional to the duration of the burst. As such, longer bursts are emphasized over shorter bursts. In some embodiments, the weight of each burst IAF is proportional to the average amplitude of the alpha wave during the burst. In some embodiments, the weight of each burst IAF is proportional to a combination of the duration of the burst and the average amplitude of the alpha wave during the burst.

At step 312, the alpha burst analyzer 106 is configured to determine the alpha burst duration of the set of EEG channels. In some embodiments, the alpha duration is calculated as the combined duration of all alpha bursts. In some embodiments, the alpha duration is compared to the EEG length (e.g., total recording time).

At step 314, the alpha burst analyzer 106 is configured to determine the EEG length of the set of EEG channels. In some embodiments, the EEG length is calculated as the recording length after artifact removal (e.g., after step 304). In some embodiments, the EEG length is calculated as the recording length before artifact removal (e.g., before step 304). In some embodiments, the EEG length is calculated as the total time duration of one or more pre-defined activities. For example, in some embodiments a pre-defined activity is a period where the eyes are closed or where the eyes are open, or a period where the person is attempting to solve a problem or move a limb, or when the person is asleep. In some embodiments, the pre-defined activity is a period where an external stimulus is presented to the subject 102, such as a light, sound, tapping sensation, vibration, electric current, or some other stimulus.

At step 316, the alpha burst analyzer 106 is configured to determine the AP of the set of EEG channels. As described above, the AP is the percentage of time in which the EEG is in an alpha burst state. In some embodiments, long and/or frequent periods of alpha waves indicate a brain which is calm, relaxed, and able to focus on activities well. It is beneficial for the brain to be in an alpha burst state than in a more random chaotic state. Therefore, in some embodiments it is beneficial to maximize AP. In some embodiments, the AP is then determined as being equal to (Alpha Duration)/(EEG Length). In some embodiments, the AP is expressed as a percentage.

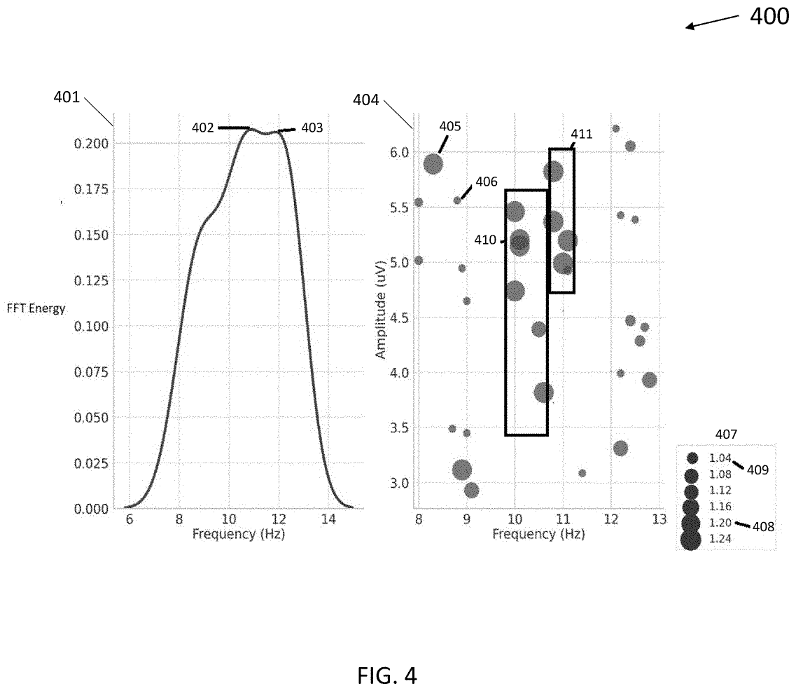

In some embodiments, the AFV and AP are presented in a report for the user (e.g., subject 102), a caregiver, or a clinician. For example, FIG. 4 illustrates an example report 400 in accordance with aspects described herein. In some embodiments, the report 400 includes a plot 404 representing the alpha bursts. In some embodiments, the size of each alpha burst is represented by a circle. As shown, an example large circle 405 corresponds to an alpha burst of approximately 1.20 seconds (e.g., as indicated by item 408 in key 407) and an example small circle 406 corresponds to an alpha burst of approximately 1.04 seconds (e.g., as indicated by item 409 in key 407). In some embodiments, the vertical position of the center of each circle represents the average amplitude of the alpha wave during the burst. In some embodiments, the average amplitude is shown in uV. In some embodiments, the plot 404 is used to identify frequencies of alpha activity. For example, by visual inspection of the plot 404, it is clear that a significant amount of alpha activity occurs at approximately 10.5 Hz (as shown by the rectangle 410) and at approximately 11.0 Hz (as shown by the rectangle 411). In some embodiments, the report 400 includes a spectral plot 401. The spectral plot 401 represents the energy of the EEG recording. In some embodiments, the spectral plot 401 corresponds to a Fourier Transform (e.g., FFT) of the EEG recording. For example, in plot 401, the energy of the EEG is shown at frequency values between approximately 6 Hz and approximately 15 Hz with peaks at approximately 10.5 Hz (indicated at 402) and at approximately 11.0 Hz (indicated at 403).

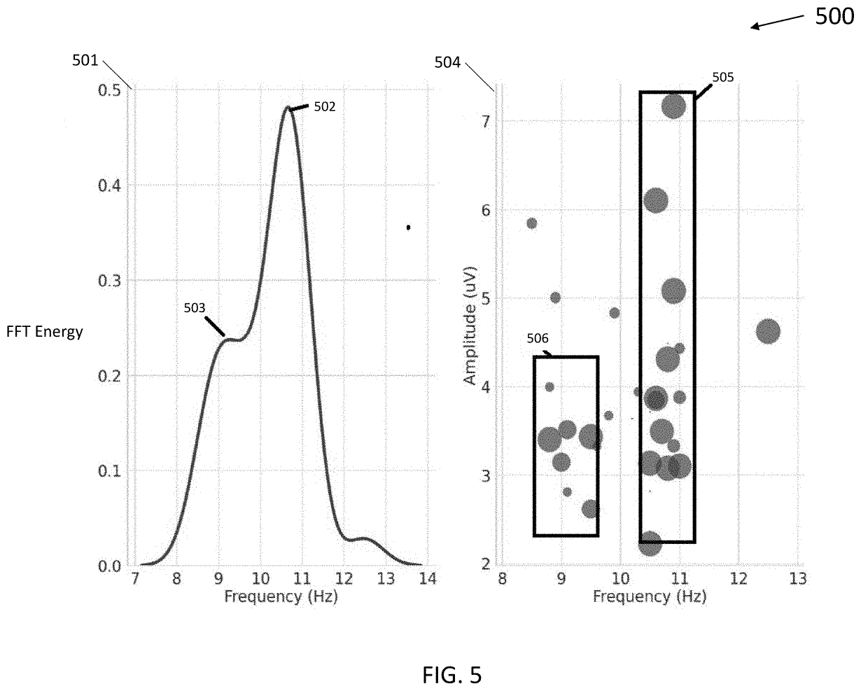

FIG. 5 illustrates another example report 500 in accordance with aspects described herein. In some embodiments, the report 500 includes a plot 504 representing the alpha bursts. As shown, an example group of larger circles is shown at approximately 10.5 Hz (as shown by the rectangle 505) and an example group of smaller circles at a lower amplitude are shown at approximately 9.5 Hz (as shown by the rectangle 506). In some embodiments, the report 500 includes a spectral plot 501. The spectral plot 501 represents the energy of the EEG recording. For example, in plot 501, the energy of the EEG is shown with peaks at approximately 10.5 Hz (indicated at 502) and at approximately 9.5 Hz (indicated at 503), matching the frequencies of alpha activity in the plot 504.

In some embodiments, brain stimulation is used to optimize the alpha burst parameters. For example, in some embodiments brain stimulation is used to minimize the AFV and/or maximize the AP of the subject 102. In some embodiments, the brain stimulation is provided to the subject 102 via the treatment device(s) 108. In some embodiments, the treatment device 108 is configured to provide one or more types of brain stimulation. In some embodiments, the brain stimulation includes providing a magnetic fields having a stimulation (or therapeutic) frequency to the head of the subject 102. In some embodiments, the treatment device 108 provides brain stimulation using alternating magnetic fields. In some embodiments, the treatment device 108 provides brain stimulation using pulsed magnetic fields. In some embodiments, the treatment device 108 provides brain stimulation using transcranial electrical stimulation. In some embodiments, the treatment device 108 provides brain stimulation using focused ultrasound. In some embodiments, the treatment device 108 provides brain stimulation using functional Near Infrared Spectroscopy (fNIRS). In some embodiments, the treatment device 108 provides brain stimulation using sensory stimulation. For example, in some embodiments the treatment device 108 provides sensory stimulation including flashing light, sound, video, or touch.

Due to the resonance of the brain, stimulation at or near the alpha frequency affects the brain through entrainment, making brain activity more rhythmic and regular. In some embodiments, brain stimulation is performed with a stimulation frequency equal to the weighted average IAF across all bursts in an EEG recording. For example, in some embodiments a recording consists of two bursts, burst₁ and burst₂. If burst₁ has an IAF of 10.0 Hz and a duration of 0.5 sec, and if burst₂ has an IAF of 11.0 Hz and a duration of 1.0 sec, the resulting weighted average IAF is [(0.5 sec)(10.0 Hz)+(1.0 sec)(11.0 Hz)]/(1.5 sec)=10.67 Hz.

In some embodiments, brain stimulation is performed with a stimulation frequency equal to the average IAF across all alpha bursts, irrespective of the burst length. For example, in some embodiments a recording consists of two bursts, burst₁ and burst₂. If burst₁ has an IAF of 10.0 Hz and burst₂ has an IAF of 11.0 Hz, then the resulting average IAF is [(10.0 Hz)+(11.0 Hz)]/2=10.5 Hz.

In some embodiments, brain stimulation is performed with a stimulation frequency that hops around during a stimulation session. In some embodiments, the EEG is recorded during stimulation and/or during breaks in stimulation. As such, in some embodiments the stimulation frequency is adjusted based upon the IAF of the most recently recorded alpha burst. In some embodiments, stimulation is paused to allow recording of the new EEG. In some embodiments, the stimulation frequency is updated for each new alpha burst recorded. In some embodiments, the stimulation frequency is updated at regular pre-determined intervals. For example, in some embodiments the stimulation frequency at each interval based upon the most recent alpha burst, an average of a pre-defined number of alpha bursts (e.g., average of last 10 alpha bursts), or a pre-defined burst duration since the last update (e.g., average of alpha bursts occurring in last 10 secs).

In some embodiments, the brain stimulation frequency is adjusted based on an optimization of the AFV, the AP, or a combination of the two. For example, in some embodiments stimulation is applied with an initial frequency, and the AFV and/or AP are determined during or following the stimulation. The stimulation frequency is adjusted up or down by a step-size (e.g., 0.01 Hz, 0.1 Hz, 1 Hz, etc.) and the AFV and/or AP is re-determined during or following the next round of stimulation. In some embodiments, the stimulation frequency is adjusted to minimize AFV and/or maximize AP. In some embodiments, optimization routines are used by the alpha burst analyzer 106 and/or the treatment device(s) 108. For example, in some embodiments optimization routines such as gradient based optimization, conjugate gradient descent optimization, Newton and quasi-newton optimization, simulated annealing, and/or mean-field annealing can be used.

In some embodiments, the alpha burst parameters of the subject 102 are compared to an expected value. For example, in some embodiments the AFV and/or AP of the subject 102 is compared to an expected or target value. In some embodiments, the expected value(s) correspond to AFV and/or AP values indicating optimal brain health. In some embodiments, the expected value(s) correspond to average values collected from a set of known healthy individuals (e.g., a database of optimal AFV and AP values). A therapeutic stimulation frequency is selected based on a result of the comparison to the expected value(s). In some embodiments, the stimulation frequency is selected to influence (e.g., optimize) the AFV and/or AP of the subject 102. In some embodiments, the stimulation frequency is selected to tune the AFV and/or AP of the subject 102 to the expected value(s).

In some embodiments, brain stimulation is used to optimize (e.g., minimize) the AFV of the subject 102 by moving the subject's AFV below a pre-defined threshold. In some embodiments, the pre-defined AFV threshold is selected based on the AFV values of known healthy individuals. For example, in one embodiment if the average AFV of healthy individuals is 0.2 Hz, the AFV threshold is set to a corresponding value (e.g., 0.2 Hz, 0.25 Hz, etc.). In some embodiments, brain stimulation is used to optimize (e.g., maximize) the AP of the subject 102 by moving the subject's AP above a pre-defined threshold. In some embodiments, the pre-defined AP threshold is selected based on the AP values of known healthy individuals. For example, in one embodiment if the average AP of healthy individuals is 30%, the AP threshold is set to a corresponding value (e.g., 30%, 25%, etc.).

In some embodiments, the treatment device 108 is configured to provide brain stimulation by generating a magnetic field that covers the entire surface of the subject's head. In some embodiments, the magnetic field that covers the entire surface of the subject's head with a substantially uniform distribution. In some embodiments, the treatment device 108 is configured to provide brain stimulation by generating a magnetic field that is directed towards a specific portion of the subject's head. For example, in some embodiments the magnetic field is directed to one or more areas of the scalp that are of interest (e.g., EEG channels of interest).

In some embodiments, alpha parameter optimization via brain stimulation is used to improve focus, concentration, mood, sleep quality, or sense of well-being. In some embodiments, alpha parameter optimization via brain stimulation is used to treat a mental disorder. For example, in some embodiments alpha parameter optimization via brain stimulation improves symptoms of Autism Spectrum Disorder, Alzheimer's disease, ADHD, schizophrenia, anxiety, depression, coma, Parkinson's disease, substance abuse, bipolar disorder, sleep disorder, eating disorder, tinnitus, traumatic brain injury, post-traumatic stress disorder, or fibromyalgia.

As described above, systems and methods for determining and optimizing alpha burst parameters are provided herein. In at least one embodiment, the alpha burst parameters include the AFV and AP of a person's brain. In some embodiments, the alpha burst parameters are optimized using targeted, personalized, brain stimulation. The AFV and AP are used as part of a report (or analysis) to estimate a person's mental state or capacity.

### Hardware and Software Implementations

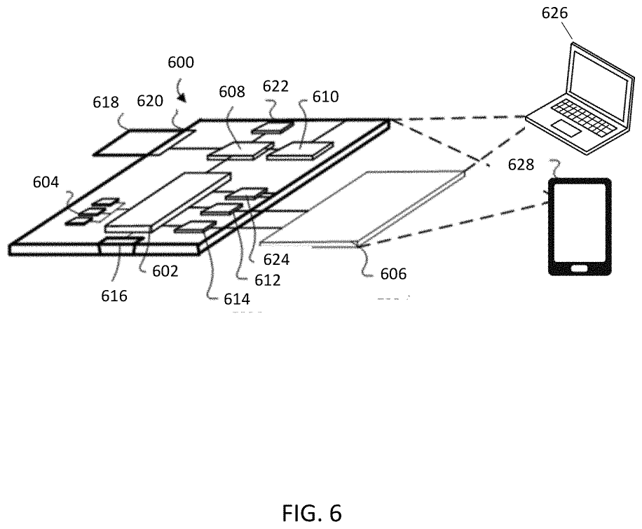

FIG. 6 shows an example of a generic computing device 600, which may be used with some of the techniques described in this disclosure (e.g., alpha burst analyzer 106, cloud application 112, or user application 114). Computing device 600 includes a processor 602, memory 604, an input/output device such as a display 606, a communication interface 608, and a transceiver 610, among other components. The device 600 may also be provided with a storage device, such as a micro-drive or other device, to provide additional storage. Each of the components 600, 602, 604, 606, 608, and 610, are interconnected using various buses, and several of the components may be mounted on a common motherboard or in other manners as appropriate.

The processor 602 can execute instructions within the computing device 600, including instructions stored in the memory 604. The processor 602 may be implemented as a chipset of chips that include separate and multiple analog and digital processors. The processor 602 may provide, for example, for coordination of the other components of the device 600, such as control of user interfaces, applications run by device 600, and wireless communication by device 600.

Processor 602 may communicate with a user through control interface 612 and display interface 614 coupled to a display 606. The display 606 may be, for example, a TFT LCD (Thin-Film-Transistor Liquid Crystal Display) or an OLED (Organic Light Emitting Diode) display, or other appropriate display technology. The display interface 614 may comprise appropriate circuitry for driving the display 606 to present graphical and other information to a user. The control interface 612 may receive commands from a user and convert them for submission to the processor 602. In addition, an external interface 616 may be provided in communication with processor 602, so as to enable near area communication of device 600 with other devices. External interface 616 may provide, for example, for wired communication in some implementations, or for wireless communication in other implementations, and multiple interfaces may also be used.

The memory 604 stores information within the computing device 600. The memory 604 can be implemented as one or more of a computer-readable medium or media, a volatile memory unit or units, or a non-volatile memory unit or units. Expansion memory 618 may also be provided and connected to device 600 through expansion interface 620, which may include, for example, a SIMM (Single In Line Memory Module) card interface. Such expansion memory 618 may provide extra storage space for device 600, or may also store applications or other information for device 600. Specifically, expansion memory 618 may include instructions to carry out or supplement the processes described above, and may include secure information also. Thus, for example, expansion memory 618 may be provided as a security module for device 600, and may be programmed with instructions that permit secure use of device 600. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 604, expansion memory 618, memory on processor 602, or a propagated signal that may be received, for example, over transceiver 610 or external interface 616.

Device 600 may communicate wirelessly through communication interface 608, which may include digital signal processing circuitry where necessary. Communication interface 608 may in some cases be a cellular modem. Communication interface 608 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 610. In addition, short-range communication may occur, such as using a Bluetooth, WiFi, or other such transceiver (not shown). In addition, GPS (Global Positioning System) receiver module 622 may provide additional navigation- and location-related wireless data to device 600, which may be used as appropriate by applications running on device 600.

Device 600 may also communicate audibly using audio codec 624, which may receive spoken information from a user and convert it to usable digital information. Audio codec 624 may likewise generate audible sound for a user, such as through a speaker, e.g., in a handset of device 600. Such sound may include sound from voice telephone calls, may include recorded sound (e.g., voice messages, music files, etc.) and may also include sound generated by applications operating on device 600. In some examples, the device 600 includes a microphone to collect audio (e.g., speech) from a user. Likewise, the device 600 may include an input to receive a connection from an external microphone.

The computing device 600 may be implemented in a number of different forms, as shown in FIG. 6. For example, it may be implemented as a computer (e.g., laptop) 626. It may also be implemented as part of a smartphone 628, smart watch, tablet, personal digital assistant, or other similar mobile device.

Some implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language resource), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending resources to and receiving resources from a device that is used by the user; for example, by sending web pages to a web browser on a user's client device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

### Terminology

The phrasing and terminology used herein is for the purpose of description and should not be regarded as limiting.

Measurements, sizes, amounts, and the like may be presented herein in a range format. The description in range format is provided merely for convenience and brevity and should not be construed as an inflexible limitation on the scope of the invention. Accordingly, the description of a range should be considered to have specifically disclosed all the possible subranges as well as individual numerical values within that range. For example, description of a range such as 1-20 meters should be considered to have specifically disclosed subranges such as 1 meter, 2 meters, 1-2 meters, less than 2 meters, 10-11 meters, 10-12 meters, 10-13 meters, 10-14 meters, 11-12 meters, 11-13 meters, etc.

Furthermore, connections between components or systems within the figures are not intended to be limited to direct connections. Rather, data or signals between these components may be modified, re-formatted, or otherwise changed by intermediary components. Also, additional or fewer connections may be used. The terms “coupled,” “connected,” or “communicatively coupled” shall be understood to include direct connections, indirect connections through one or more intermediary devices, wireless connections, and so forth.

Reference in the specification to “one embodiment,” “preferred embodiment,” “an embodiment,” “some embodiments,” or “embodiments” means that a particular feature, structure, characteristic, or function described in connection with the embodiment is included in at least one embodiment of the invention and may be in more than one embodiment. Also, the appearance of the above-noted phrases in various places in the specification is not necessarily referring to the same embodiment or embodiments.

The use of certain terms in various places in the specification is for illustration purposes only and should not be construed as limiting. A service, function, or resource is not limited to a single service, function, or resource; usage of these terms may refer to a grouping of related services, functions, or resources, which may be distributed or aggregated.

Furthermore, one skilled in the art shall recognize that: (1) certain steps may optionally be performed; (2) steps may not be limited to the specific order set forth herein; (3) certain steps may be performed in different orders; and (4) certain steps may be performed simultaneously or concurrently.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The term “about” as used in the specification and the claims (e.g., “X has a value of about Y” or “X is about equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of” “only one of” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

It will be appreciated to those skilled in the art that the preceding examples and embodiments are exemplary and not limiting to the scope of the present disclosure. It is intended that all permutations, enhancements, equivalents, combinations, and improvements thereto that are apparent to those skilled in the art upon a reading of the specification and a study of the drawings are included within the true spirit and scope of the present disclosure. It shall also be noted that elements of any claims may be arranged differently including having multiple dependencies, configurations, and combinations.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A method of treating a subject, comprising:
obtaining or having obtained measurement data representative of a plurality of alpha bursts associated with the subject's brain, the plurality of alpha bursts corresponding to successive bursts of activity in the subject's brain between 8-13 Hz;
determining or having determined a plurality of intrinsic frequencies of the subject's brain based on the measurement data representative of the plurality of alpha bursts;
selecting a stimulation frequency based on the plurality of intrinsic frequencies of the subject's brain; and
providing a stimulation treatment at the stimulation frequency close to a head of the subject to influence at least one alpha burst parameter of the subject's brain.

2. The method of claim 1, wherein providing the stimulation treatment close to the head of the subject to influence the at least one alpha burst parameter includes optimizing at least one of an alpha frequency variability (AFV) and an alpha prevalence (AP) of the subject.

3. The method of claim 2, wherein optimizing the AFV of the subject includes minimizing the AFV of the subject.

4. The method of claim 2, wherein optimizing the AP of the subject includes maximizing the AP of the subject.

5. The method of claim 1, wherein obtaining the measurement data representative of the plurality of alpha bursts includes collecting an electroencephalogram (EEG) of the subject.

6. The method of claim 1, wherein selecting the stimulation frequency includes calculating an average frequency of the plurality of intrinsic frequencies.

7. The method of claim 6, wherein the average frequency is a weighted average corresponding to a duration of each alpha burst associated with the plurality of intrinsic frequencies.

8. The method of claim 1, further comprising:
determining or having determined a value of the at least one alpha burst parameter based on the measurement data representative of the plurality of alpha bursts;
comparing the value of the at least one alpha burst parameter to an expected value for the at least one alpha burst parameter;
selecting, based on a result of the comparison, a tuning frequency; and
providing a second stimulation treatment at the tuning frequency close to the head of the subject to influence at the least one alpha burst parameter of the subject's brain.

9. The method of claim 1, further comprising:
obtaining or having obtained measurement data representative of a second plurality of alpha bursts associated with the subject's brain;
determining or having determined a second plurality of intrinsic frequencies of the subject's brain based on the measurement data representative of the second plurality of alpha bursts;
selecting a second stimulation frequency based on the second plurality of intrinsic frequencies of the subject's brain;
providing a second stimulation treatment at the second stimulation frequency close to the head of the subject to influence at the least one alpha burst parameter of the subject's brain.

10. A system for providing treatment to a subject, comprising:
at least one stimulation source;
at least one memory storing computer-executable instructions; and
at least one processor for executing the instructions stored on the memory, wherein execution of the instructions programs the at least one processor to perform operations comprising:
obtaining measurement data representative of a plurality of alpha bursts associated with the subject's brain, the plurality of alpha bursts corresponding to successive bursts of activity in the subject's brain between 8-13 Hz;
determining a plurality of intrinsic frequencies of the subject's brain based on the measurement data representative of the plurality of alpha bursts;
selecting a stimulation frequency based on the plurality of intrinsic frequencies of the subject's brain; and
providing, via the at least one stimulation source, a stimulation treatment at the stimulation frequency close to a head of the subject to influence at least one alpha burst parameter of the subject's brain.

11. The system of claim 10, wherein providing the stimulation treatment close to the head of the subject to influence the at least one alpha burst parameter includes optimizing at least one of an alpha frequency variability (AFV) and an alpha prevalence (AP) of the subject.

12. The system of claim 11, wherein optimizing the AFV of the subject includes minimizing the AFV of the subject.

13. The system of claim 11, wherein optimizing the AP of the subject includes maximizing the AP of the subject.

14. The system of claim 10, wherein obtaining the measurement data representative of the plurality of alpha bursts associated with the subject's brain includes collecting an electroencephalogram (EEG) of the subject.

15. The system of claim 10, wherein selecting the stimulation frequency includes calculating an average frequency of the plurality of intrinsic frequencies.

16. The system of claim 15, wherein the average frequency is a weighted average corresponding to a duration of each alpha burst associated with the plurality of intrinsic frequencies.

17. The system of claim 10, wherein execution of the instructions further programs the at least one processor to perform operations comprising:
determining a value of the at least one alpha burst parameter based on the measurement data associated with the plurality of alpha bursts;
comparing the value of the at least one alpha burst parameter to an expected value for the at least one alpha burst parameter;
selecting, based on a result of the comparison, a tuning frequency; and
providing, via the at least one stimulation source, a second stimulation treatment at the tuning frequency close to the head of the subject to influence the at least one alpha burst parameter of the subject's brain.

18. The system of claim 10, wherein execution of the instructions further programs the at least one processor to perform operations comprising:
obtaining measurement data representative of a plurality of second alpha bursts associated with the subject's brain;
determining a second plurality of intrinsic frequencies of the subject's brain based on the measurement data representative of the second plurality of alpha bursts;
selecting a second stimulation frequency based on the second plurality of intrinsic frequencies of the subject's brain; and
providing, via the at least one stimulation source, a second stimulation treatment at the second stimulation frequency close to the head of the subject to influence the at least one alpha burst parameter of the subject's brain.

19-40. (canceled)

41. The method of claim 2, wherein optimizing the AFV of the subject includes moving the AFV of the subject below a pre-defined AFV threshold.

42. The method of claim 41, wherein the pre-defined AFV threshold is derived from AFV values corresponding to a plurality of healthy individuals.

43. The method of claim 2, wherein optimizing the AP of the subject includes moving the AP of the subject above a pre-defined AP threshold.

44. The method of claim 43, wherein the pre-defined AP threshold is derived from AP values corresponding to a plurality of healthy individuals.

45. The system of claim 11, wherein optimizing the AFV of the subject includes moving the AFV of the subject below a pre-defined AFV threshold.

46. The system of claim 45, wherein the pre-defined AFV threshold is derived from AFV values corresponding to a plurality of healthy individuals.

47. The system of claim 11, wherein optimizing the AP of the subject includes moving the AP of the subject above a pre-defined AP threshold.

48. The system of claim 47, wherein the pre-defined AP threshold is derived from AP values corresponding to a plurality of healthy individuals.

49. The method of claim 8, wherein determining the value of the at least one alpha burst parameter includes determining a value of at least one of an alpha frequency variability (AFV) and an alpha prevalence (AP) of the subject.

50. The system of claim 17, wherein determining the value of the at least one alpha burst parameter includes determining a value of at least one of an alpha frequency variability (AFV) and an alpha prevalence (AP) of the subject.
